(19) United States
(12) Patent Application Publication
(10) Pub. No.: US 2003/0064061 A1
(43) Pub. Date: Apr. 3, 2003

(54) Bcr-Abl oligomerization domain polypeptides and uses therefor

(75) Inventors: Xun Zhao, San Diego, CA (US); Saghi Ghaffari, Boston, MA (US); Harvey F. Lodish, Brookline, MA (US); Vladimir N. Malashkevich, Revere, MA (US); Peter S. Kim, Bryn Mawr, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA (US)

(21) Appl. No.: 10/193,651

(22) Filed: Jul. 9, 2002

Related U.S. Application Data
(60) Provisional application No. 60/303,857, filed on Jul. 9, 2001.

(51) Int. Cl. A61K 38/57; C07H 21/04; C12N 9/99; C12P 21/02; C12N 5/06

(52) U.S. Cl. 424/94.1; 435/69.1; 435/184; 435/320.1; 435/325; 536/23.2

(57) ABSTRACT

Recombinant Bcr-Abl polypeptides that form a stable α-helical structure; nucleic acids encoding recombinant Bcr-Abl polypeptides; methods of identifying or designing inhibitors of Bcr-Abl oligomerization and methods of inhibiting Bcr-Abl oligomerization in cells and in individuals in need of inhibiting Bcr-Abl oligomerization, such as individuals who have developed or are at risk of developing chronic myelogenous leukemia or acute lymphoblastic leukemia.

### RELATED APPLICATIONS

This application claims the benefit of U.S. Provisional Application No. 60/303,857 filed Jul. 9, 2001. The entire teachings of the above-referenced application are incorporated herein by reference in their entirety.

### GOVERNMENT SUPPORT

The invention was supported, in whole or in part, by grants PO1 GM56552 and PO 1 HL 32262 from National Institutes of Health. The Government has certain rights in the invention.

### BACKGROUND OF THE INVENTION

The Bcr-Abl oncogene is essential in the development of over 95% of chronic myelogenous leukemia (CML) and 20% of acute lymphoblastic leukemia (ALL) (A. Gotoh, H. E. Broxmeyer, Curr Opin Hematol 4, 3-11 (1997); C. L. Sawyers, N Engl J Med 340, 1330-40 (1999). Bcr-Abl is formed by the fusion of the breakpoint cluster region (Bcr) gene on chromosome 22 with the proto-oncogene c-Abl on chromosome 9 in a reciprocal translocation event that results in the formation of the Philadelphia (Ph) chromosome (C. L. Sawyers, N Engl J Med 340:1330-40(1999)). c-Abl encodes a nonreceptor tyrosine kinase, the activity of which is tightly regulated in normal cells (A. S. Ponticelli, C. A. Whitlock, N. Rosenberg, O. N. Witte, Cell 29:953-60 (1982)). Fusion of Bcr sequences upstream of c-Abl constitutively activates Abl tyrosine kinase and is essential for Bcr-Abl transformation (A. M. Pendergast, A. J. Muller, M. H. Havlik, Y. Maru, O. N. Witte, Cell 66:161-71 (1991); J. R. McWhirter, J. Y. Wang, Mol Cell Biol 11:1553-65 (1991)) (FIG. 1). A method of interfering with fusion of the Bcr sequences upstream of c-Abl and, thus, of inhibiting such transformation would be useful.

### SUMMARY OF THE INVENTION

The invention described herein is related, at least in part, to the determination of the crystal structure of the Bcr-Abl oligomerization domain and the modes of Bcr-Abl oligomer formation which result in transformation of Bcr-Abl expressing cells. It has also been determined that Bcr-Abl oligomerization is inhibited in a dominant negative manner, with the result that the transforming potential of Bcr-Abl is also inhibited, thus demonstrating the therapeutic potential of inhibiting Bcr-Abl oligomerization in reducing proliferation and oncogenesis of Bcr-Abl expressing cells. The crystal structure of the Bcr-Abl oligomerization domain, understanding of the process by which Bcr-Abl oligomerization occurs and recombinant Bcr₁₇₂ described herein provide methods and compositions useful in inhibiting and in identifying or designing inhibitors of Bcr-Abl oligomerization.

Described herein are isolated forms of the Bcr-Abl oligomerization domain; nucleic acids, e.g., DNA, RNA, that encode the recombinant forms of the oligomerization domain; vectors that contain and express nucleic acids that encodes recombinant oligomerization domains; cells, such as prokaryotic cells, e.g., bacteria, and eukaryotic cells, particularly mammalian, e.g., human cells, that contain the vectors and in which the recombinant oligomerization domains are expressed; and antibodies that bind recombinant oligomerization domains of the present invention. Also described herein are methods of expressing recombinant oligomerization domains; methods of inhibiting Bcr-Abl oligomerization and, thus, Bcr-Abl transformation of eukaryotic cells, e.g., mammalian cells, such as human cells; methods of identifying or designing agents or drugs that inhibit Bcr-Abl oligomerization; agents or drugs identified or designed by the methods of the present invention and methods of inhibiting Bcr-Abl oligomerization and, thus, Bcr-Abl transformation of cells/constitutive activation of Abl tyrosine kinase activity, in an individual in need of such inhibition, e.g., an individual, such as a human, who has developed or is at risk of developing CML or ALL.

Accordingly, in one aspect, the invention is directed to an isolated Bcr-Abl oligomerization domain polypeptide. In one embodiment, the Bcr-Abl oligomerization domain polypeptide forms a stable α-helical structure. In another embodiment, the Bcr-Abl oligomerization domain polypeptide is a Bcr₁₋₇₂ sequence. In particular embodiments, the polypeptide has the amino acid sequences of SEQ ID NO: 1, SEQ ID NO: 3, SEQ ID NO: 5, SEQ ID NO: 7 or SEQ ID NO: 9.

In another aspect, the invention is directed to isolated nucleic acids encoding Bcr-Abl oligomerization domain polypeptides. In one embodiment, the nucleic acid encodes a Bcr-Abl oligomerization domain polypeptide that forms a stable α-helical structure. In another embodiment, the nucleic acid encodes a Bcr-Abl oligomerization domain polypeptide that is a Bcr₁₋₇₂ sequence. In particular embodiments, the nucleic acid encodes a Bcr-Abl oligomerization domain that has the amino acid sequence of SEQ ID NO: 1, SEQ ID NO: 3, SEQ ID NO: 5, SEQ ID NO: 7 or SEQ ID NO: 9. In particular embodiments, the nucleic acid has the nucleotide sequence of SEQ ID NO: 2, SEQ ID NO: 4, SEQ ID NO: 6, SEQ ID NO: 8 or SEQ ID NO:10.

In another aspect, the invention is directed to a vector comprising an isolated nucleic acid molecule of the invention operatively linked to a regulatory sequence, e.g., an expression vector, as well as to a recombinant host cell comprising the vector. The invention also provides a method for producing a Bcr-Abl oligomerization domain polypeptide encoded by an isolated nucleic acid molecule described herein, comprising culturing a recombinant host cell of the invention under conditions suitable for expression of the nucleic acid molecule. The method can further comprise isolating the Bcr-Abl oligomerization domain polypeptide.

In another aspect, the invention is directed to a method for identifying agents which bind to a Bcr-Abl oligomerization domain comprising contacting an isolated Bcr-Abl oligomerization domain with a candidate agent and detecting the resulting domain-agent complex. A candidate agent can be a protein, polypeptide, peptidomimetic, prodrug, binding agent, antibody, small molecule or other drug, or ribozyme. In particular embodiments, the Bcr-Abl oligomerization domain has the amino acid sequence of SEQ ID NO: 1, SEQ ID NO: 3, SEQ ID NO: 5, SEQ ID NO: 7 or SEQ ID NO: 9.

In another aspect, the invention is directed to a method for identifying agents which inhibit, e.g., partially or completely, Bcr-Abl oligomerization. In one embodiment, the invention relates to a method of identifying an agent that inhibits the transformation of cells. The method comprises introducing Bcr-Abl and a candidate inhibitor into cells, e.g, test cells; maintaining the cells under conditions appropriate for Bcr-Abl transformation of the cells to occur; and comparing the extent to which transformation of the cells occurs in the presence of the candidate inhibitor to the extent to which transformation of the cells occurs in the absence of the inhibitor. A candidate inhibitor can be a protein, polypeptide, peptidomimetic, prodrug, binding agent, antibody, small molecule or other drug, or ribozyme. In particular embodiments, the Bcr-Abl oligomerization domain has the amino acid sequence of SEQ ID NO: 1, SEQ ID NO: 3, SEQ ID NO: 5, SEQ ID NO: 7 or SEQ ID NO: 9. The cells can be mammalian cells. The agents identified by the methods of the invention are also within the scope of the invention.

In another aspect, the invention is directed to a method for identifying agents which inhibit, e.g., partially or completely, the formation of a stable α-helical structure. The method comprises introducing a Bcr-Abl oligomerization domain that forms a stable α-helical structure and a candidate inhibitor into cells, e.g, test cells; maintaining the cells under conditions appropriate for formation of a stable α-helical structure; and comparing the extent to which the formation of a stable α-helical structure occurs in the presence of the candidate inhibitor to the extent to which formation of a stable α-helical structure occurs in the absence of the inhibitor. If the extent of formation of a stable α-helical structure is decreased in the presence of the candidate inhibitor, then an inhibitor has been identified. A candidate inhibitor can be a protein, polypeptide, peptidomimetic, prodrug, binding agent, antibody, small molecule or other drug, or ribozyme. In particular embodiments, the Bcr-Abl oligomerization domain has the amino acid sequence of SEQ ID NO: 1, SEQ ID NO: 3, SEQ ID NO: 5, SEQ ID NO: 7 or SEQ ID NO: 9. The cells can be mammalian cells. The agents identified by the methods of the invention are also within the scope of the invention.

In another aspect, the invention is directed to an antibody, or an antigen-binding fragment thereof, which specifically binds to a polypeptide of the invention. The antibody can be, for example, a polyclonal or a monoclonal antibody.

In yet another aspect, the invention is directed to methods of treating or preventing a disease associated with the Bcr-Abl oncogene in a subject in need thereof comprising administering an effective amount of a Bcr-Abl oligomerization domain inhibitor to the subject. In particular embodiments, the invention is directed to methods of treating or preventing CML and ALL. The method can further comprise administering a tyrosine kinase inhibitor.

In another aspect, the invention is directed to a method of preparing a medicament for use in treating or preventing a disease associated with a Bcr-Abl oncogene in a subject, the medicament comprising a Bcr-Abl oligomerization domain inhibitor. In particular embodiments, the disease is CML or ALL. The medicament can further comprise a tyrosine kinase inhibitor.

### BRIEF DESCRFPTION OF THE DRAWINGS

The file of this patent contains at least one drawing in color. Copies of this patent with color drawings will be provided by the Patent and Trademark office upon request and payment of the necessary fee.

FIG. 1 is a schematic representation of the structural and functional domains of Bcr, Abl, and Bcr-Abl (C. M. Verfaillie, Hematol Oncol Clin North Am 12:1-29 (1998)). In addition to the other domains contained in the two genes, the two domains essential for transforming activity—OLI from Bcr and TK from Abl—are depicted. Depending on the site of the breakpoint in the Bcr gene, the fusion protein formed by the fused Bcr-Abl gene can vary in size from 190-230 kDa. Each fusion protein contains the same portion of the c-Abl protein, but differs in the length of the Bcr portion. The breakpoints in Bcr in CML and ALL are indicated by arrowheads.

The sequence of wild-type Bcr₁₋₇₂ (SEQ ID NO.: 3) is indicated, with the sequence of the coiled-coil region (Bcr₃₀₋₆₅) (SEQ ID NO: 1) underlined. Letters immediately above the sequence indicate the heptad repeat (a) and (d) positions. Heptad repeats—a repeated seven-amino acid pattern, denoted (a-g)ₙ with hydrophobic residues at the first (a) and fourth (d) positions—are a key feature of coiled-coil sequences (A. D. McLachlan, M. Stewart, L. B. Smillie, J Mol Biol 98:281-91 (1975); A. Lupas, M. Van Dyke, J. Stock, Science 252:1162-4 (1991); B. Berger, et al., Proc Natl Acad Sci USA 92:8259-63 (1995)). Cylinders below the sequence indicate the α1-helix (residues 5-15) (SEQ ID NO: 11; SEQ ID NO: 12) and the a2-helix (residues 28-67) (SEQ ID NO: 13; SEQ ID NO: 14) observed in the crystal structure.

FIGS. 2A and 2B show the extensive interactions in the dimer interface.

FIG. 2A shows that in the dimer, the α2-helix of one monomer (blue) is sandwiched between the α1 and α2-helices of a second monomer (yellow), and vice versa. The side chains of (a) and (d) residues in the coiled coil and the residues in the aromatic core are displayed. The residues in the coiled-coil region and the aromatic core are well defined in electron density maps (average main-chain B-factor is approximately 30 Å²), whereas residues beyond the interface area and those from the loop are quite flexible (average main-chain B-factors is approximately 53 Å²). The aromatic core is boxed. Inset shows an expanded image of the MAD-phased electron density contoured at 1.2σ in the area of the aromatic core. For a clearer view, Phe 15 is not shown.

FIG. 2B shows the surface electrostatic potential of one monomer, with its dimeric partner shown in green. Basic regions are blue and acidic regions are red. Nearly 4200 Å² of the solvent-accessible surface is buried upon dimer formation. The figure was prepared using MOLSCRIPT (P. J. Kraulis, J Appl Crystallogr 24:946-50 (1991)) and GRASP (A. Nicholls, K. A. Sharp, B. Honig, Proteins 11:281-96 (1991)).

FIGS. 3A and 3B show packing of Bcr₁₋₇₂ tetramers with two orthogonal views. The tetramer has an approximate 222-point symmetry, with three two-fold rotation axes intersecting near Leu45. The four monomers are colored in yellow, blue, dark purple, and light purple, respectively.

FIG. 3A is a top view looking down the flat surface of helix bundles in dimers.

FIG. 3B is a side view showing dimer-dimer stacking.

FIGS. 4A and 4B show dimer-dimer interface. Approximately 3400 Å² of the solvent-accessible surface is buried upon the association of two dimmers. Shown are two views of the surface electrostatic potential of one dimer, with the other dimer displayed in a ribbon-style representation. Basic regions on the surface are blue and acidic regions are red. The side chains of the residues involved in dimer-dimer interactions are shown.

FIG. 4A is a front view of the highly hydrophobic surface involved in the association with another dimer.

FIG. 4B is a back view showing the highly hydrophilic surface that is solvent exposed.

FIG. 5 is a graphic representation of results of a Rat-1 focus-formation assay. Data shown are the average of three dishes and are representative of four independent experiments. Error bars indicate the standard deviation of foci numbers between the experiments.

FIGS. 6A, 6B and 6C show the nucleic acid and amino acid sequences of the human Bcr gene.

FIGS. 6A and 6B show the nucleotide sequence of the human Bcr gene (SEQ ID NO: 22) which contains 4739 base pairs with the coding region beginning at nucleotide 489 and continuing to nucleotide 4304. This sequence is reported as Accession Number X02596 (gi:29420).

FIG. 6C shows the amino acid sequence of the human Bcr protein (SEQ ID NO: 21) which contains 1271 amino acid residues. This sequence is reported as Accession Number P11274 in the Swissprotein database (gi:114887).

### DETAILED DESCRIPTION OF THE INVENTION

The invention described herein is related, at least in part, to the determination of the crystal structure of the N-terminal Bcr-Abl oligomerization domain and the modes of Bcr-Abl oligomer formation (which result in transformation of cells). It has also been determined that Bcr-Abl oligomerization is inhibited in a dominant negative manner, with the result that the transforming potential of Bcr-Abl is also inhibited, thus demonstrating the therapeutic potential of inhibiting Bcr-Abl oligomerization in reducing proliferation and ontogenesis of Bcr-Abl expressing cells. The crystal structure of the Brc-Abl oligomerization domain, understanding of the process by which Bcr-Abl oligomerization occurs and recombinant Bcr₁₇₂ described herein provide methods and compositions useful in inhibiting and in identifying or designing inhibitors of Bcr-Abl oligomerization.

In one aspect, the invention is related to an isolated or purified Bcr-Abl oligomerization domain polypeptide, particularly an isolated or purified Bcr-Abl oligomerization domain polypeptide that forms a stable α-helical structure. An “isolated” or “purified” (e.g., partially or substantially) polypeptide is in a form that is distinct from the form in which it occurs in nature. In one embodiment, the polypeptide is part of a composition (crude extract). In another embodiment, the polypeptide is substantially free of cellular material or other contaminating proteins from the cell or tissue source from which the Bcr-Abl oligomerization domain polypeptide is derived, or substantially free from chemical precursors or other chemicals when chemically synthesized. The language “substantially free of cellular material” includes preparations of Bcr-Abl oligomerization domain polypeptides in which the polypeptide is separated from cellular components of the cells from which it is isolated, recombinantly produced or chemically synthesized. Such preparations of Bcr-Abl oligomerization domain polypeptides have less than about 30%, 20%, 10%, 5% (by dry weight) of chemical precursors or non-Bcr-Abl oligomerization domain chemicals. Various methods, such as gel electrophoresis or chromatography can be used to identify polypeptides that are substantially free of cellular material. The polypeptide can be fused to other sequences, and still be considered isolated or purified.

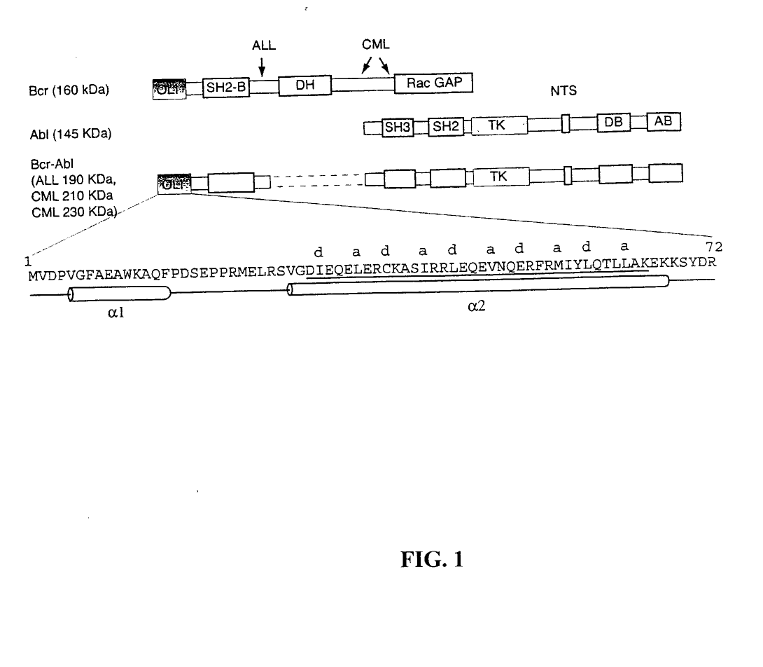

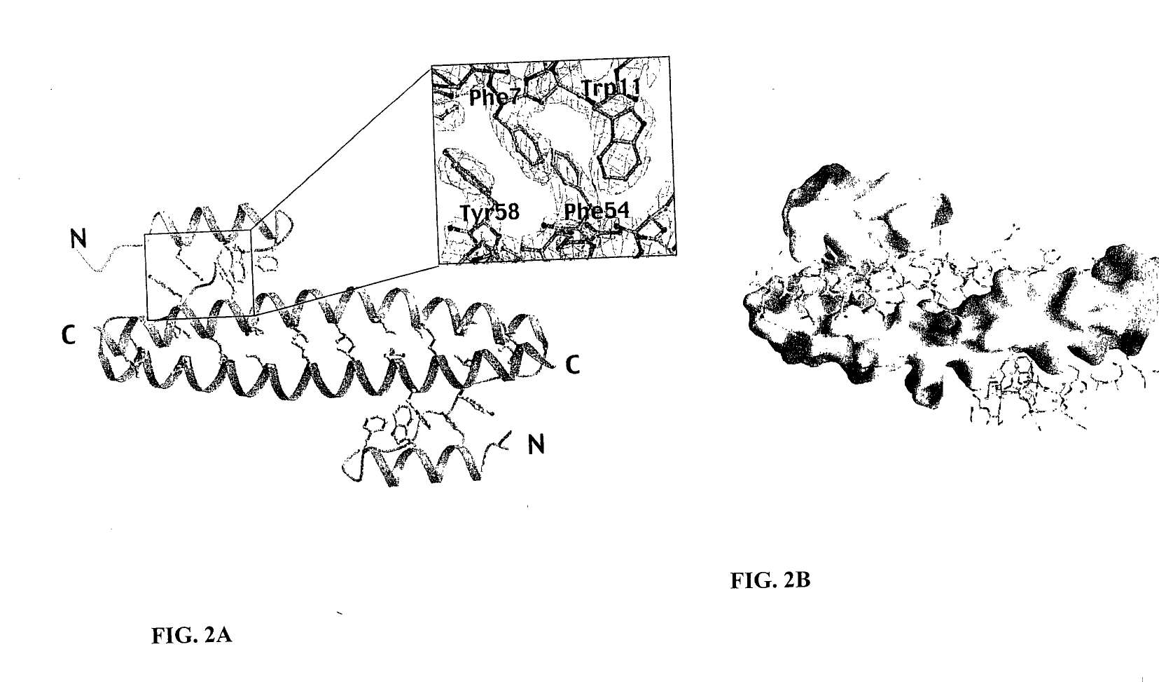

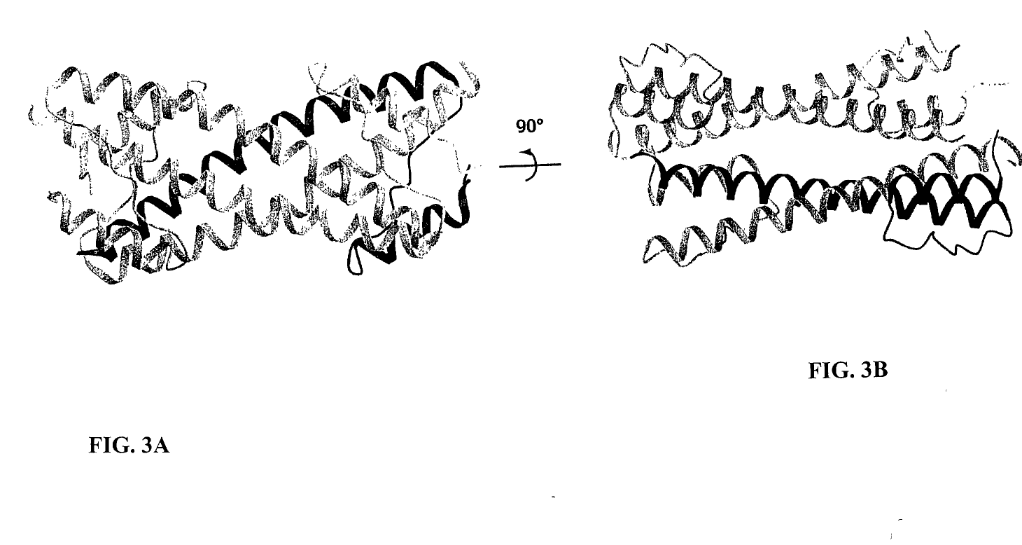

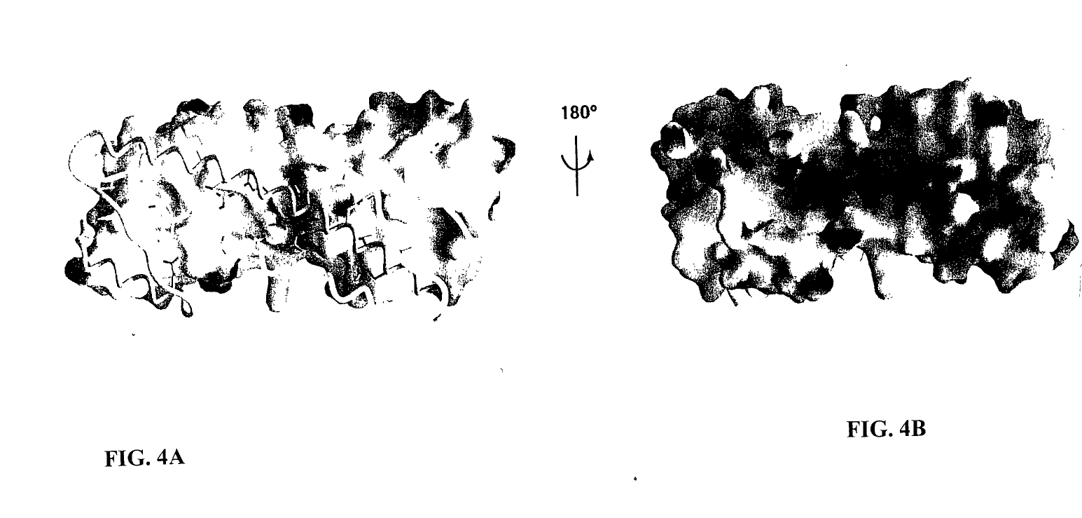

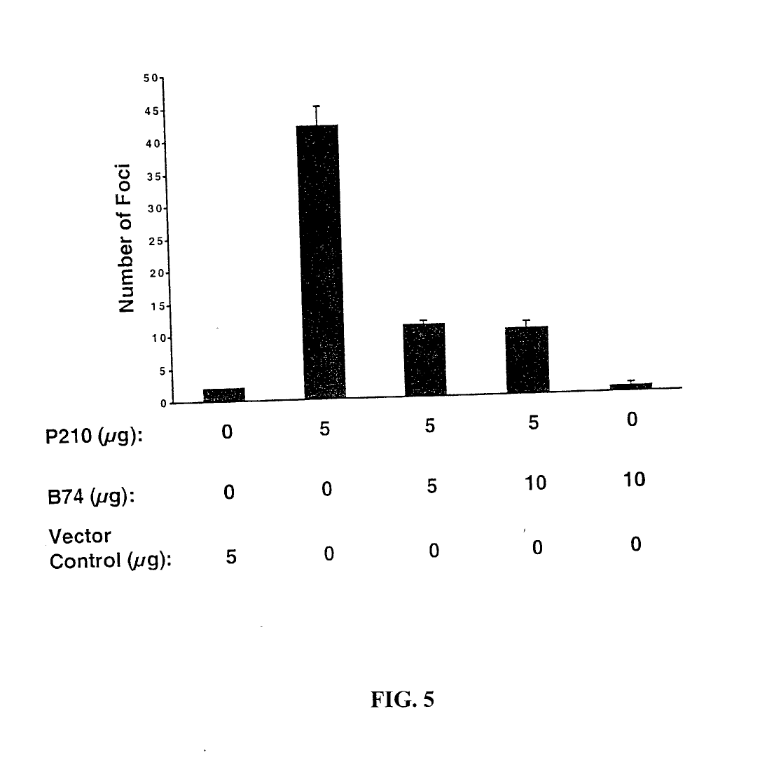

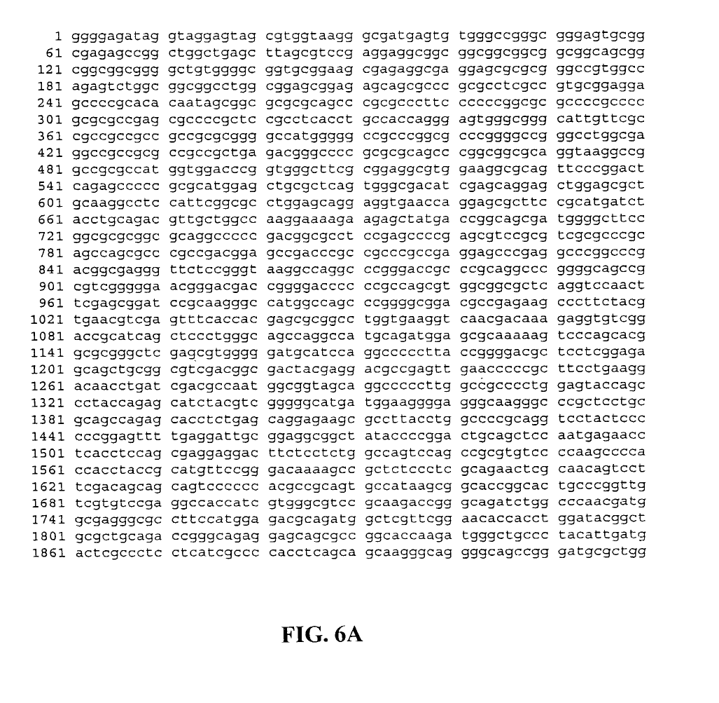

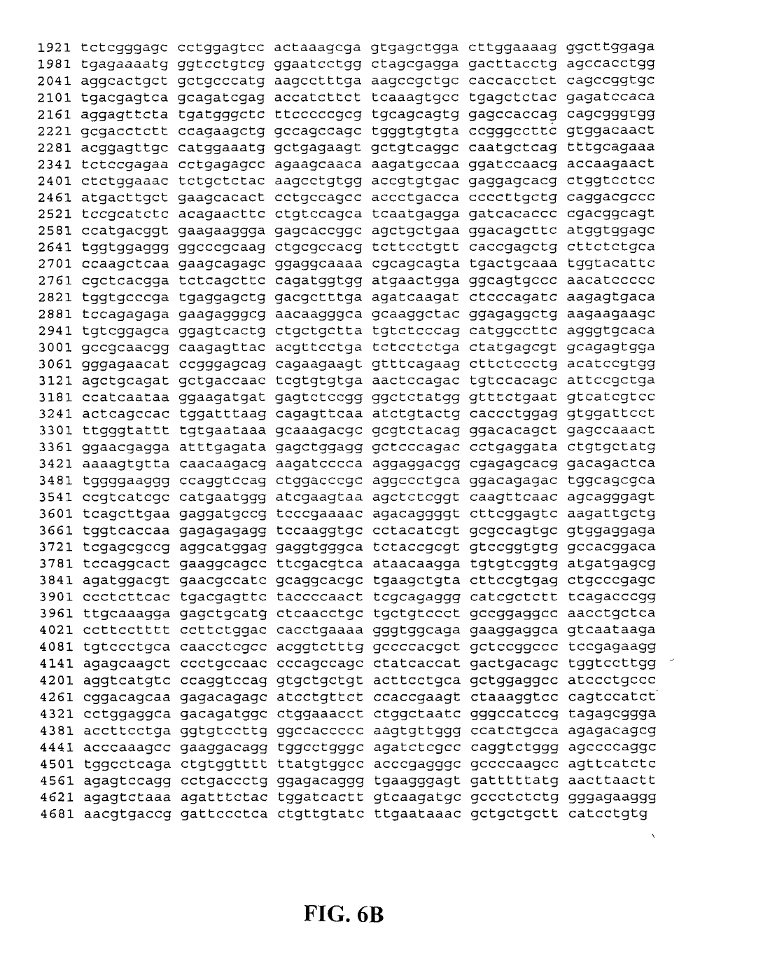

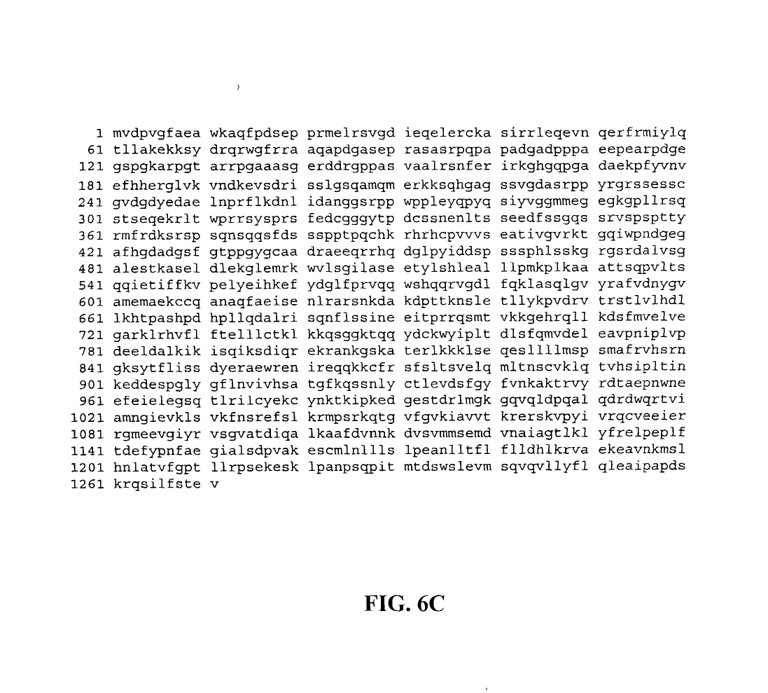

As shown in FIG. 1, the human Bcr protein contains 1271 amino acids and multiple domains including: OLI, the oligomerization domain; SH2-B, the SH2 binding regions; DH, the domain homologous to the human Dbl and yeast Cdc24 proteins; and RacGAP, the domain with GTPase-activating activity for RAC. The c-Abl protein contains 1097 amino acids including: SH3/SH2, Scr-homology domains 3/2; TK, tyrosine kinase domain; NTS, nuclear translocalization signal; DB, DNA binding domain; and AB, action-binding motif. Two domains, OLI from Bcr and TK from Abl, are essential for transforming activity. Depending on the site of the breakpoint in the Bcr gene, the fusion protein expressed by the Bcr-Abl fused gene can vary in size from 190-230 kDa. Each fusion protein contains the same portion of the c-Abl protein, but differs in the length of the Bcr portion.

The phrase “Bcr-Abl oligomerization domain” as used herein refers to an N-terminal portion of the Bcr protein, or an allele or variant thereof, which contains the amino acid residues essential for Abl kinase activation. The wild-type sequences reported in the database entry with Accession Number P 11274 (SWISS-PRTO) and the corresponding X02596 form the bases for the sequences described herein. A Bcr-Abl oligomerization domain can contain only the essential residues, e.g., Bcr₃₀₋₆₅, which contains amino residues 30 through 65 of the Bcr protein (also known as the coiled-coil region) (SEQ ID NO: 1). Typically, however, additional amino acid residues from the N-terminal portion of the Bcr protein are included, particularly those adjacent to the essential residues. Those additional residues are known to increase the solubility and/or stability of the polypeptide. Any number of these additional residues can be included in an isolated Bcr-Abl oligomerization domain, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 additional residues adjacent to the amino acid residue at position 30 or the amino acid residue at position 65 or both those adjacent to the amino acid residue at position 30 and at position 65 of SEQ ID NO: 1 can be included. In particular, it is desirable for the Bcr-Abl oligomerization domain polypeptide to form an α-helix. For example, a number of Bcr oligomerization domains contain a Bcr₁₋₇₂ sequence. A “Bcr₁₋₇₂ sequence” as used herein refers to an amino acid sequence containing the amino residues at positions 1-72 of the N-terminus of the wild-type Bcr protein sequence, or any allele or variant thereof. Wild-type Bcr₁₋₇₂ (SEQ ID NO.:3) is indicated in FIG. 1, with the sequence of the coiled-coil region (Bcr₃₀₋₆₅) (SEQ ID NO: 1) underlined. Letters immediately above the sequence indicate the heptad repeat a and d positions. Heptad repeats—a repeated seven-amino acid pattern, denoted (a-g)ₙ with hydrophobic residues at the first (a) and fourth (d) positions—are a key feature of coiled-coil sequences (A. D. McLachlan, M. Stewart, L. B. Smillie, J Mol Biol 98:281-91 (1975); A. Lupas, M. Van Dyke, J. Stock, Science 252:1162-4 (1991); B. Berger, et al., Proc Natl Acad Sci USA 92:8259-63 (1995)). Cylinders below the sequence indicate the α1 helix (residues 5-15) (SEQ ID NO: 11; SEQ ID NO: 12) and the α2-helix (residues 28-67) (SEQ ID NO: 13; SEQ ID NO: 14) observed in the crystal structure. Another Bcr₁₋₇₂ (SEQ ID NO.:5) was produced in which one residue, the cysteine at position 38 was modified to an alanine.

In addition, a number of Bcr oligomerization domains contain a Bcr₁₋₇₄ sequence. A “Bcr₁₋₇₄ sequence” as used herein refers to an amino acid sequence containing the amino residues at positions 1-74 of the N-terminus of the wild-type Bcr protein sequence, or any allele or variant thereof. Wild-type Bcr₁₋₇₄ (SEQ ID NO.:7) also contains the sequence of the coiled-coil region (Bcr₃₀₋₆₅) (SEQ ID NO: 1), and the other domains noted for the wild-type Bcr₁₋₇₂ sequence (SEQ ID NO: 3). Another Bcr₁₋₇₄ (SEQ ID NO.:9) was produced in which one residue, the cysteine at position 38 was modified to an alanine.

Other Bcr-Abl oligomerization domain polypeptides of the present invention are substantially identical to SEQ ID NO:1, SEQ ID NO: 3, SEQ ID NO: 5, SEQ ID NO: 7 and SEQ ID NO: 9 and retain the functional activity of the polypeptides of SEQ ID NO:1, SEQ ID NO: 3, SEQ ID NO: 5, SEQ ID NO: 7 and SEQ ID NO: 9, yet differ in amino acid sequence due to natural allelic variation or mutagenesis. A useful Bcr-Abl oligomerization domain polypeptide includes an amino acid sequence at least about 45%, preferably 55%, 65%, 75%, 85%, 95%, 98 or 99% identical to the amino acid sequences of SEQ ID NO:1, SEQ ID NO: 3, SEQ ID NO: 5, SEQ ID NO: 7 and SEQ ID NO: 9 and retains the functional activity of the Bcr-Abl oligomerization domain polypeptides of SEQ ID NO:1, SEQ ID NO: 3, SEQ ID NO: 5, SEQ ID NO: 7 and SEQ ID NO: 9. In other instances, the Bcr-Abl oligomerization domain polypeptide has an amino acid sequence 55%, 65%, 75%, 85%, 95%, 98% or 99% identical to the Bcr-Abl oligomerization domain. In one embodiment, the Bcr oligomerization domain polypeptide retains a functional activity of the Bcr oligomerization polypeptide of SEQ ID NO:1.

To determine the percent identity of two amino acid sequences or of two nucleic acids, the sequences are aligned for optimal comparison purposes, wherein gaps are introduced in the sequences being compared. The amino acid residues at corresponding amino acid positions or nucleotides at corresponding nucleotide positions are then compared. When a position in a first sequence is occupied by the same amino acid residue or nucleotide as the corresponding position in a second sequence, then the molecules are identical at that position. The percent identity between the two sequences is a function of the number of identical positions shared by the sequences (e.g., % identity=# of identical positions/total # of positions×100).

As described herein, the determination of percent homology between two sequences can be accomplished using a mathematical algorithm. Examples of a mathematical algorithm utilized for the comparison of two sequences is the algorithm of Karlin and Altschul (1993) Proc. Nat'l Acad. Sci. USA 90: 5873-5877 and the algorithm incorporated into the NBLAST and XBLAST programs of Altschul, et al. (1990) J. Mol. Biol. 215:403-410. Another example of a mathematical algorithm utilized for the comparison of sequences is the algorithm of Myers and Miller, CABIOS (1989).

The invention also provides Bcr-Abl oligomerization domain chimeric or fusion proteins. As used herein, a Bcr-Abl oligomerization domain “chimeric protein” or “fusion protein” comprises a Bcr-Abl oligomerization domain polypeptide fused in-frame to an additional component (a non-Bcr-Abl oligomerization domain polypeptide). Within a Bcr-Abl oligomerization domain fusion protein, the Bcr-Abl oligomerization domain polypeptide can correspond to all or a portion of a Bcr-Abl oligomerization domain polypeptide, preferably at least a portion that forms a stable α-helical structure. The additional component can be fused to the N-terminus or C-terminus of the Bcr-Abl oligomerization domain polypeptide. An example of a fusion protein is a GST-Bcr-Abl oligomerization domain fusion protein in which the Bcr-Abl oligomerization domain sequences are fused to the C-terminus of the GST sequences. Such fusion proteins can facilitate the purification of recombinant Bcr-Abl oligomerization domain polypeptide. Another example of a fusion protein is a Bcr-Abl oligomerization domain-immunoglobulin fusion protein in which all or part of a Bcr-Abl oligomerization domain polypeptide is fused to sequences derived from a member of the immunoglobulin protein family. The Bcr-Abl oligomerization domain-immunoglobulin fusion proteins of the invention can be used as immunogens to produce anti-Bcr-Abl oligomerization domain antibodies in a subject, to purify Bcr-Abl oligomerization domain ligands and in screening assays to identify molecules which inhibit the interaction of Bcr-Abl oligomerization domain with a Bcr-Abl oligomerization domain ligand.

A Bcr-Abl oligomerization domain chimeric or fusion protein of the invention can be produced by standard recombinant DNA techniques. For example, DNA fragments coding for the different polypeptide sequences are ligated together in-frame in accordance with conventional techniques (e.g., using blunt-ended or stagger-ended termini for ligation, restriction enzyme digestion, and enzymatic ligation). In another embodiment, conventional techniques such as an automated DNA synthesizer can be used. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A Bcr-Abl oligomerization domain encoding nucleic acid can be cloned into such an expression vector such that the fusion moiety is linked in-frame to the Bcr-Abl oligomerization domain polypeptide.

As described in detail herein and as depicted in FIGS. 1, 2A, 2B, 3A, 3B, 4A and 4B, two N-shaped monomers of the Bcr-Abl oligomerization domain dimerize by swapping N-terminal helices and by forming an antiparallel coiled-coil between C-terminal helices. Two of the resulting dimers stack onto each other to form a tetramer. The N-terminal portion of Bcr containing the Bcr-Abl oligomerization domain is required for Abl kinase activation (J. R. McWhirter, D .L. Glasso, J. Y. Wang, Mol Cell Bio 13:7587-95 (1993); X. Zhang., R Subrahmanyam, R. Wong. A. W. Gross, R. Ren, Mol Cell Biol 21:840-53 (2001)).

In another aspect, the present invention is directed to isolated nucleic acids encoding recombinant Bcr-Abl oligomerization domains. As used herein, “nucleic acids” include DNA, e.g, cDNA, genomic DNA, a gene, RNA, e.g., mRNA, and analogs thereof. DNA molecules can be single-stranded or double-stranded. Single-stranded RNA or DNA can be the coding, or sense, strand, or the non-coding, or antisense, strand. The nucleic acid can include all or a portion of the coding strand and can further comprise additional non-coding sequneces such as introns and non-coding 5′ and 3′ sequences, e.g., regulatory sequences. In addition, the nucleic acid can be linked to a marker sequence, for example, a sequence that encodes a polypeptide to assist in isolation or purification of the polypeptide. Such sequences include, for example, those which encode a glutathione-S-transerase (GST) fusion protein and those which encode a hemagglutinin A (HA) polypeptide marker from influenza. As used herein, an “isolated” nucleic acid is one that is separated from other nucleic acids with which it is normally associated in its natural state, and/or which has been partially or completely purified away from other transcribed sequences, e.g., those contained in a library. Thus, an isolated nucleic acid of the invention can be substantially isolated with respect to the complex cellular milieu in which it naturally occurs, with respect to culture medium when produced by recombinant techniques, or with regard to chemical precursors or other chemicals when chemically synthesized. In some instances, the isolated nucleic acid forms a portion of a composition, e.g., a pharmaceutical composition. In other instances, the isolated material is purified to essential homogeneity, for example, as determined by PAGE or column chromatography, such as HPLC. With regard to genomic DNA, the term isolated can also refer to nucleic acid which is separated from the chromosome with which the genomic DNA is normally associated in its natural state.

The nucleic acid can be fused to other coding or regulatory sequences, but would still be considered isolated. Thus, recombinant nucleic acid contained in a vector is encompassed by the definition of isolated used herein. Isolated nucleic acid molecules also include recombinant nucleic molecules contained in heterologous host cells, as well as partially or completely purified DNA molecules in solution. Isolated nucleic acids also encompass in vivo and in vitro RNA transcripts of the DNA of the present invention.

Such nucleic acids can encode, for example, a Bcr₃₀₋₆₅ (SEQ ID NO: 1), a Bcr₁₋₇₄ (for example, SEQ ID NO: 7 or SEQ ID NO: 9), and various Bcr₁₋₇₂ (for example, SEQ ID NO: 3 or SEQ ID NO: 5). In one embodiment, the nucleic acid encodes a recombinant Bcr₁₋₇₂ that forms a stable α-helical structure, with a Tₘ of 84° C. In an alternate embodiment, the nucleic acid encodes a Bcr₁₋₇₂ in which the Cys38 (cysteine residue present at position 38 of the Bcr-Abl oligomerization domain as shown in FIG. 1, which is SEQ ID NO.: 3) is mutated to or replaced by alanine. The latter Bcr₁₋₇₂ sequence is SEQ ID NO.: 5. In another embodiment, the nucleic acid encodes a recombinant Bcr₁₋₇₄ that forms a stable α-helical structure. In an alternate embodiment, the nucleic acid encodes a Bcr₁₋₇₄in which the Cys38 (cysteine residue present at position 38 of the Bcr-Abl oligomerization domain of the wild-type Bcr₁₋₇₄ (SEQ ID NO.: 7) is mutated to or replaced by alanine. The mutated Bcr₁₋₇₄ sequence is SEQ ID NO.: 9.

A related aspect of the invention pertains to nucleic acid constructs containing a nucleic acid encoding an amino acid sequence of the invention. The constructs comprise a vector, e.g., an expression vector, into which a sequence of the invention has been inserted in a sense or antisense orientation. “Vector” as used herein refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced, e.g., bacterial vectors having a bacterial origin of replication and episomal mammalian vectors. Other vectors, e.g., non-episomal mammalian vectors, are integrated into the genome of a host cell upon introduction into the host cell, and are thereby replicated along with the host genome. Moreover, certain vectors, known as expression vectors, are capable of directing the expression of genes to which they are operably linked.

Particular recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. In particular embodiments, the expression vector comprises nucleic acids which encode SEQ ID NO:1, SEQ ID NO: 3, SEQ ID NO: 5, SEQ ID NO: 7 and SEQ ID NO: 9. In particular embodiments, these nucleic acids are SEQ ID NO: 2, SEQ ID NO: 4, SEQ ID NO: 6, SEQ ID NO: 8 and SEQ ID NO: 10. Within a recombinant expression vector, “operably linked” or “operatively linked” means that the nucleotide sequence of interest is linked to the regulatory sequence in a manner which allows for expression of the nucleotide sequence, e.g., in an in vitro transcription/translation system or in a host cell when the vector is introduced into the host cell. The term “regulatory sequence” includes promoters, enhancers and other expression control elements, e.g., polyadenylation signals. Such regulatory sequences are described, for example, in Goeddel, “Gene Expression Technology”, Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells, e.g., tissue-specific regulatory sequences. It will be appreciated by those skilled in the art that the design of the expression vector can depend on such factors as the choice of the host cell to be transformed and the level of expression of polypeptide desired.

Another related aspect of the invention pertains to host cells into which a recombinant expression vector of the invention has been introduced. Cells, such as bacterial cells and eukaryotic cells, e.g., mammalian cells, particularly human cells, that contain (have incorporated therein) an expression vector comprising nucleic acids encoding a recombinant Bcr of the present invention and in which the encoded product is expressed is a further subject of the invention. In particular embodiments, a host cell of the present invention comprises a nucleic acid that encodes the amino acid sequence of SEQ ID NO:1, SEQ ID NO: 3, SEQ ID NO: 5, SEQ ID NO: 7 or SEQ ID NO: 9. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells, e.g., E. coli, insect cells, yeast or mammalian cells, such as Chinese hamster ovary cells (CHO), COS cells or human cells. Other suitable host cells are known to those skilled in the art.

Vector DNA can be introduced into prokaryotic or eukaryotic cells via conventional transfection techniques. As used herein, the term “transfection” is intended to refer to a variety of art-recognized techniques for introducing a foreign nucleic acid molecule, e.g., DNA, into a host cell, including calcium phosphate or calcium chloride co-precipitation, DEAE-dextran-mediated transfection, lipofection, or electroporation. Suitable methods for transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker, e.g., for resistance to antibiotics, can also be introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection, e.g., cells that have incorporated the selectable marker gene survive, while the other cells die.

A host cell of the invention, such as a prokaryotic or eukaryotic host cell in culture, can be used to produce, i.e., express, a Bcr-Abl oligomerization domain polypeptide of the invention. Accordingly, the invention further provides methods for producing a Bcr-Abl oligomerization domain polypeptide using the host cells of the invention. In one embodiment, the method comprises culturing the host cell of the invention, into which a recombinant expression vector encoding a Bcr-Abl oligomerization domain polypeptide of the invention has been introduced, in a suitable medium such that the polypeptide is produced. In another embodiment, the method further comprises isolating the Bcr-Abl oligomerization domain polypeptide from the medium or the host cell.

In another aspect, the invention is directed to methods for identifying an agent, e.g., protein, polypeptide, peptidomimetic, prodrug, binding agent, antibody, small molecule or other drug, or ribozyme which binds to Bcr-Abl oligomerization domains. In one embodiment, the invention is directed to a method for identifying an agent which binds to SEQ ID NO:1, SEQ ID NO: 3, SEQ ID NO: 5, SEQ ID NO: 7 or SEQ ID NO: 9 or an allele or variant thereof. Methods for screening agents, e.g., candidate binding agents or test binding agents which bind to Bcr-Abl oligomerization domains as well as the agents identified by the methods are included within the scope of the invention. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other approaches are applicable to polypeptide, non-peptide oligomer or small molecule libraries of compounds (Lam, K. S., Anticancer Drug Des. 12:145 (1997)).

In one embodiment, the Bcr-Abl oligomerization domain polypeptide can be contacted with an agent to be tested. The level or amount of binding is assessed, either directly or indirectly, for example by detection of an domain-agent complex. If a domain-agent complex is detected, then the agent is an agent that binds to the polypeptide.

Determining the ability of the test agent to bind to the polypeptide can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product.

It may be desirable to immobilize either the polypeptide, the agent, or other components of the method on a solid support, in order to facilitate separation of complexed from uncomplexed forms of the polypeptide, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtiter plates, test tubes, and micro-centrifuge tubes.

In yet another aspect, the invention is directed to a method of identifying inhibitors of Bcr-Abl oligomerization, also referred to as inhibitors of constitutive activation of Abl tyrosine kinase. It may be desirable to utilize agents identified as binding to Bcr-Abl oligomerization domains in these methods. The method comprises introducing Bcr-Abl into appropriate cells, referred to as test cells, (e.g., by transfecting the cells with a vector from which Bcr-Abl is expressed), in combination with/in the presence of a candidate inhibitor under conditions suitable for Bcr-Abl transformation of cells to occur, determining the extent to which transformation of the cells occurs; and comparing the extent to which Bcr-Abl transformation occurred in the presence of the candidate inhibitor to the extent to which Bcr-Abl transformation occurred in control cells, wherein if test cells are transformed to a lesser extent than are control cells, the candidate inhibitor is an inhibitor of Bcr-Abl oligomerization.

“Control” cells as used herein refers to cells that are of the same type or of a similar type as the corresponding test cells in any particular study. Control cells are maintained under conditions identical to those of the corresponding test cells, the sole difference in conditions between test cells and control cells being the absence of the candidate inhibitor in the control cells. Cells for use in the methods of the invention can be of any type, but are typically mammalian cells such as human fibroblasts or Rat-1 cells. “Transform” as used herein refers to a cell's proliferative or oncogenetic characteristics.

In one embodiment, the method of identifying an inhibitor of a Bcr-Abl oligomerization domain polypeptide is a focus-formation assay in which appropriate cells, termed “test” cells, for example, Rat-1 cells, are transfected with a vector expressing Bcr-Abl. The term “Bcr-Abr” when used to describe an expression product of the Bcr-Abl fusion gene refers to a full-length wild-type Bcr-Abl, an allele or variant thereof, or a sufficient portion of a Bcr-Abl expression product to effect transformation of the cells under appropriate conditions. The term “transformation” as used herein refers to the changing of a cell from it normal state to a state in which it has at least one characteristic of a proliferative cell. The test cells can, for example, be transfected using a retroviral expression vector comprising DNA encoding Bcr-Abl, such as pMSCV-210-IRES-GFP in the presence of a candidate inhibitor. The resulting transfected cells are maintained under conditions appropriate for Bcr-Abl transformation of cells to occur. The extent to which transformation occurs in the test cells is determined and compared to the extent to which transformation occurred in control cells. If the extent to which transformation occurs in the test cells is less than the extent to which it occurs in the control cells, the candidate inhibitor is an inhibitor of Bcr-Abl transformation of cells.

The extent of the transformation of the cells can be determined by any method known to those of skill in the art. For example, the extent of the transformation of the cells can be determined by microscopic examination of the cells to determine the presence of characteristics consistent with proliferation or oncogenesis. If test cells exhibit fewer characteristics consistent with proliferation or oncogenesis than do the corresponding control cells, then the candidate inhibitor is an inhibitor of Bcr-Abl transformation of cells. Alternatively, or in addition to microscopic examination of the cells, the extent of transformation of the cells can be determined by quantifying the amount of Abl tyrosine kinase produced by the cells. If test cells exhibit a decreased production of Abl tyrosine kinase compared to the production of Abl tyrosine kinase by the corresponding control cells, then the candidate inhibitor is an inhibitor of Bcr-Abl transformation of cells.

In another aspect, the invention is directed to a method for identifying agents which inhibit, e.g., partially or completely, the formation of a stable α-helical structure. The method comprises introducing a Bcr-Abl oligomerization domain that forms a stable α-helical structure and a candidate inhibitor into cells, e.g, test cells; maintaining the cells under conditions appropriate for formation of a stable α-helical structure; and comparing the extent to which the formation of a stable α-helical structure occurs in the presence of the candidate inhibitor to the extent to which formation of a stable α-helical structure occurs in the absence of the inhibitor. If the extent of formation of a stable α-helical structure is decreased in the presence of the candidate inhibitor, then an inhibitor has been identified. A candidate inhibitor can be a protein, polypeptide, peptidomimetic, prodrug, binding agent, antibody, small molecule or other drug, or ribozyme. In particular embodiments, the Bcr-Abl oligomerization domain has the amino acid sequence of SEQ ID NO: 1, SEQ ID NO: 3, SEQ ID NO: 5, SEQ ID NO: 7 or SEQ ID NO: 9. The cells can be mammalian cells. The agents identified by the methods of the invention are also within the scope of the invention.

In another aspect, the present invention is directed to inhibitors of Bcr-Abl transformation of cells, particularly inhibitors of Bcr-Abl transformation of human cells. Such inhibitors are also referred to herein as inhibitors of Bcr-Abl oligomerization and as inhibitors of constitutive activation of Abl tyrosine kinase activity. Inhibitors can be, for example, proteins, polypeptides, peptidomimetics, interfering RNA, (e.g., RNAi), prodrugs, binding agents, antibodies, small molecules, or other drugs or ribozymes. In one embodiment, the candidate inhibitor is a product, e.g., protein or proteinaceous product, encoded by DNA or RNA present in an expression vector that is expressed in test cells. Candidate inhibitors for use in the methods of the invention can be present in/obtained from, for example, libraries such as chemical libraries, e.g., combinatorial libraries or libraries of biological compounds; culture media; plant parts and tissues or cells obtained from a wide variety of animals, including humans.

In a particular embodiment, inhibitors are recombinant Bcr_{30-65,} Bcr₁₋₇₂, Bcr₁₋₇₄ or alleles or variants thereof formed, for example, by the addition, deletion, substitution or alteration of at least one amino acid residue. Such inhibitors can be naturally occurring or synthetic or a combination of the two.

In another embodiment, inhibitors of the present invention are antibodies or antigen binding fragments thereof that specifically bind the Bcr-Abl oligomerization domain. Both polyclonal and monoclonal antibodies that specifically bind the Bcr oligomerization domain are included, although monoclonal antibodies are typically preferred. In one embodiment, the antibody or antigen binding fragment specifically binds to Bcr-Abl oligomerization domain polypeptides encoded by an isolated nucleic acid that encodes SEQ ID NO:1, SEQ ID NO: 3, SEQ ID NO: 5, SEQ ID NO: 7 or SEQ ID NO: 9. An isolated Bcr-Abl oligomerization domain polypeptide or a portion or fragment thereof, can be used as an immunogen to generate antibodies that bind Bcr-Abl oligomerization domains using standard techniques for polyclonal or monoclonal antibody preparation. The Bcr-Abl oligomerization domain polypeptides or antigenic peptide fragments can be used as immunogens. For example, an antigenic peptide of a Bcr-Abl oligomerization domain can comprise at least about 10, 12, 15, or 20 amino acid residues of the amino acid sequence shown in SEQ ID NO:1, SEQ ID NO: 3, SEQ ID NO: 5, SEQ ID NO: 7 and SEQ ID NO: 9 and encompass an epitope of the Bcr-Abl oligomerization domain such that an antibody raised against the peptide forms a specific immune complex with Bcr-Abl oligomerization domain polypeptides.

The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4:72 (1983)), the EBV-hybridoma technique (Cole et al., Monoclonal Antibodies and Cancer Therapy, Alan R. Liss, 1985, Inc., pp. 77-96) or trioma technique. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981). Moreover, the ordinarily skilled worker will appreciate that there are many variations of such methods that also would be useful.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In addition to acting as inhibitors of Bcr-Abl oligomerization, antibodies of the invention, e.g., a monoclonal antibody, can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Detection of the antibody can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention further pertains to agents identified by the above-described methods. Accordingly, it is within the scope of this invention to further use an agent identified as described herein in an appropriate animal model. For example, an agent identified as described herein, e.g., a test agent that is a binding agent can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A further embodiment of the present invention is a method of inhibiting or reducing, partially or completely, Bcr-Abl oligomerization and, thus, Bcr-Abl transformation of cells/constitutive activation of Abl tyrosine kinase activity, in an individual, such as a human, in need of such inhibition, e.g., an individual, such as a human, in whom Bcr-Abl oligomerization may occur or has occurred. Thus, the method can be used to either treat or prevent a disease associated with Bcr-Abl transformation. In a particular embodiment, the invention is a method of inhibiting or reducing Bcr-Abl oligomerization and thus, Bcr-Abl transformation of cells/constitutive activation of Abl tyrosine kinase activity, in an individual who has CML or ALL or is at risk of developing CML or ALL. In the method, an inhibitor of Bcr-Abl oligomerization is administered to the individual by an appropriate route and in a therapeutically sufficient dose or an in an effective amount, e.g,. an amount that is effective to inhibit or reduce Bcr-Abl transformation in the individual.

The Bcr-Abl oligomerization domain polypeptides, the nucleic acids encoding the Bcr-Abl oligomerization domain polypeptides, anti-Bcr-Abl oligomerization domain polypeptide antibodies and other inhibitors (also referred to herein as “active compounds”) of the invention can be incorporporated into pharmaceutical compositions suitable for administration. Methods of preparing medicaments containing these active compounds for treatment of Bcr-Abl oligomerization domain associated diseases are included within the scope of the invention. Typically, such medicaments contain suitable pharmaceutically acceptable carriers. Such carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy, rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agent which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of a susceptibility to a disease or condition associated with a protein kinase, and should be decided according to the judgment of a practitioner and each patient's circumstances. Effective doses may be extrapolated from dose-response curves derived from in vitro or animal model test systems.

The present invention is further illustrated by the following examples, which are not intended to be limiting in any way. (In the following examples, when two sequence identifiers follow a sequence, the first refers to the amino acid sequence, the second to the encoding nucleotide sequence.)

### Exemplification

### EXAMPLE 1

### Preparation of Bcr-Abl Oligomerization Domain Peptides

Using optimal codons for E. coli expression (S. C. Makrides, Micrbiol. Rev. 60: 512-38 (1996)), a synthetic gene sequence, denoted Bcr₁₋₇₄ (SEQ ID NO: 7), was constructed that encoded residues 1-74 of the human Bcr gene (SWISS-PROT:P11274). A Factor Xa cleavage site was incorporated upstream of the coding sequence. The constructed gene was inserted into the BamHI-HindII restriction sites of the expression vector pMALc2x (New England Biolabs), from which Bcr₁₋₇₄ (SEQ ID NO: 7) can be expressed as a chimera with E. coli maltose binding protein (MBP). The plasmid pMBP/Bcr₁₋₇₄ was expressed in E. coli TOP 10 F′ competent cells. Cells were lysed by French Press and the lysate was clarified by centrifugation at 30,000 g for 1 h at 4° C., and the fusion protein was purified using amylose agarose (New England Biolabs). Beads were suspended in Factor Xa cleavage buffer (50 mM Tris-HCl, pH 8.0, 100 mM NaCl, 2 mM CaCl₂). Factor Xa was added at a 1:500 weight:weight ratio of protease to fusion protein and the reaction incubated for 2 days at room temperature. During cleavage, approximately half of the Bcr₁₋₇₄ (SEQ ID NO: 7) protein was further trimmed to Bcr₁₋₇₂ (SEQ ID NO: 3). The two fragments, Bcr₁₋₇₄ (SEQ ID NO: 7) and Bcr₁₋₇₂ (SEQ ID NO: 3), were readily separated by reverse-phase high-performance liquid chromatography (HPLC) (Waters, Inc.) using a Vydac C18 preparative column (Vydac) with a water/acetonitrile gradient of 0.1%/min in the presence of 0.1% trifluoroacetic acid. Peak fractions were verified by mass spectrometry and lyophilized. Bcr₃₀₋₆₅ (SEQ ID NO: 1) was synthesized on a Perkin Elmer Model 431 A peptide synthesizer using the method described (D. J. Lockhart, P. S. Kim, Science 257, 947-51 (1992)). All peptides had an acetylated N-terminus and a C-terminal amide. Peptides were purified by reverse-phase HPLC and lyophilized.

### EXAMPLE 2

### Characterizing Bcr-Abl Oligomerization Domain Peptides

Bcr₁₋₇₂ (SEQ ID NO: 3) was expressed and characterized and its X-ray crystal structure determined to reveal the molecular details of Bcr-Abl oligomerization and to provide a structural basis for inhibitor design (FIG. 1).

Initially, several variants of the oligomerization domain were characterized. Previous studies indicated a coiled-coil region for residues 28-68 (SEQ ID NO: 15; SEQ ID NO: 16) (J. R. McWhirter, D. L. Glasso, J. Y. Wang, Mol Cell Biol 13:7587-95 (1993)); therefore, whether this region was sufficient for oligomerization was first assessed. The coiled-coil segment (residues 30-65, or Bcr₃₀₋₆₅) (FIG. 1) (SEQ ID NO: 1), produced by standard peptide synthesis (see Example 1), formed a stable α-helical structure with a midpoint of thermal denaturation (Tₘ) of 60° C. However, Bcr₃₀₋₆₅ (SEQ ID NO: 1) had poor solubility in physiological buffers. Notably, residues beyond the coiled-coil segment were shown to provide additional structural stability and solubility. The recombinant Bcr₁₋₇₂ (SEQ ID NO: 3) formed a stable α-helical structure with a midpoint of thermal denaturation (Tₘ) of 84° C.

Circular dichroism experiments were performed on an Aviv 62A DS circular dichroism spectrometer. Measurements from 200-260 m were performed in a 10 μM solution of protein sample in PBS (50 mM Na phosphate, 150 mM NaCl, pH 7.4) in a 1-mm path-length cuvette. Thermal denaturation was performed in a 10 mm path-length cuvette and samples were heated from 0-98° C., with equilibration times of 2 minutes and an averaging time of 1 minute. Protein concentrations were determined by absorbance at 280 nm in 20 mM phosphate-buffered 6M guanidine-hydrochloride (pH 6.5) (H. Edelhoch, Biochemistry 6, 1948-54 (1967)).

A Bcr₁₋₇₄C38A mutant gene fragment (Cys38 mutated to Ala) (SEQ ID NO: 9) was constructed by primer-mediated recombinant PCR mutagenesis (R. Higuchi, B. Krummel, R. K. Saiki, Nucleic Acids Res 16, 7351-67 (1988)). The PCR product was inserted into the MBP-expression vector pMALc2x. pMBP/Bcr₁₋₇₄C38A plasmids were transformed into E. coli JM109 competent cells for protein expression. The purification scheme for the mutant protein was the same as for wild-type. In the final step, two fragments—Bcr₁₋₇₄C38A and Bcr₁₋₇₂C38A-were purified and lyophilized. For selenomethionyl (SeMet) protein expression, the pMBP/Bcr₁₋₇₄C38A vector and LacI^{q} gene carrying pREP4 plasmids were co-transformed into E. coli strain DL41 (W. A. Hendrickson, J. R. Horton, D. M. LeMaster, EMBO J 9, 1665-72 (1990)). Large-scale cell growth was carried out as described (W. Yang, W. A. Hendrickson, E. T. Kalman, R. J. Crouch, J Biol Chem 265, 13553-9 (1990)). SeMet protein was purified by a procedure similar to that of the native protein, except 10 mM DTT or β-mecaptoethanol was added to each digestion and purification step. Diffraction-quality crystals were obtained when Cys 38 was mutated to Ala in Bcr₁₋₇₂ (SEQ ID NO: 5). The X-ray crystal structure was solved by using multi-wavelength anomalous diffraction (MAD) (W. A. Hendrickson, Science 254, 51-8 (1991)) data from a selenomethionine (Semet)-substituted Bcr₁₋₇₂ crystal and refined the structure to 2.2-Å resolution. The asymmetric unit of the Bcr₁₋₇₂ crystals contains eight equivalent protein chains that form two identical tetramers. This observation is consistent with previous biochemical studies, where tetramers were found for both the oligomerization domain and the full-length Bcr-Abl (J. R. McWhirter, D. L. Galasso, J. Y. Wang, Mol Cell Biol 13: 7587-95 (1993; A. M. Pendergast, r. Clark, E. S. Kawasaki, F. P. McCormick, O. N. Witte, Oncogene 4:759-66 (1989)). Each monomer consists of a short N-terminal helix (residues 5-15, referred to as α1; SEQ ID NO: 11: SEQ ID NO: 12), a flexible loop (residues 16-27; SEQ ID NO: 17: SEQ ID NO: 18), and a long C-terminal helix (residues 28-67, referred to as a2; SEQ ID NO: 13; SEQ ID NO: 14) (FIG. 1). Together these form an N-shaped structure with the loop allowing the two helices of one monomer to assume a parallel orientation (FIG. 2A).

The best diffracting crystals grew from 1 μL of 10 mg/ml Bcr₁₋₇₂C38A sample added to 1.0 μL of reservoir buffer containing ˜2.5 M ammonium sulfate and allowed to equilibrate against 500 μL of reservoir buffer. Crystals were in space group P2₁ (a=36.31 Å, b=122.12 Å, c=60.45 Å; a=γ=90°, β=93.24°). SeMet-Bcr₁₋₇₂ C38A was crystallized in ˜1.8M ammonium sulfate and crystals were isomorphous to wild-type protein. Prior to data collection, native and SeMet-Bcr₁₋₇₂C38A crystals were transferred into paraffin oil and flash-frozen using X-stream (Molecular Structure Corporation) or Oxford Cryosystems cryogenic crystal coolers. Initial data were collected on a Rigaku RU300 rotating-anode X-ray generator mounted on an R-axis IV area detector (Molecular Structure Corporation). Final native and MAD data sets for Bcr₁₇₂ were collected at the Howard Hughes Medical Institute Beamline X4A at Brookhaven National Laboratory using a ADSC Quantum-IV detector. For MAD data, the peak and inflection wavelengths of the selenium K absorption edge as well as the remote wavelength were selected based on the fluorescence spectrum of the SeMet-Bcr₁₋₇₂ crystal. Data sets were collected in 20° batches, allowing each batch to be collected at each wavelength before moving to the next in order to minimize crystal decay between data sets. Reflections were integrated and scaled with the programs DENZO and SCALEPACK (Z. Otwinowski, in Data Collection and Processing, L. Sawer, N. Isaacs, S. Bailey, Eds. (SERC, Daresbury Laboratory, Warrington England, pp. 55-62 (1993)). Further diffraction data processing, phase determination and map calculations were performed using the CCP4 software suite CCP4 (The CCP4 suite: programs for protein crystallography (Acta Crystallogr D50, 760-3 (1994)). Intensities were reduced to amplitudes with program TRUNCATE, and the data sets for the wavelengths closest to the selenium K absorption edge (λ1, λ2) were scaled with SCALEIT (Acta Crystallogr D50, 760-3 (1994)) to the remote wavelength (λ3) data set. MAD phases were generated with the program MLPHARE (Acta Crystallogr D50, 760-3 (1994)) from 20 selenium atoms located within the asymmetric unit using the program SOLVE (T. C. Terwiliger, J. Berendzen, Acta Crystallogr D55, 849-61 (1999)). Phases were improved and extended to higher resolution with the program DM (K. D. Cowtan, P. Main, Acta Crystallogr. D54, 487-93 (1998)). Electron density map interpretation and model building were done with the program O (T. A. Jones, J. W. Zou, S. Cowan, M. Kjeldgaard, Acta Crystallogr D47, 110-9 (1991)). The structure of Bcr₁₇₂ was refined at 2.2 Å resolution using the program CNS (A. T. Brunger et al. Acta Crystallogr D54, 905-21 (1998)). The correctness of the structure was checked with simulated annealing omit maps and with the program PROCHECK (R. A. Laskowski, M. W. MacArthur, D. S. Moss, J. M. Thornton, J Appl Crystallogr. 26, 283-91(1993)). All residues of Bcr₁₋₇₂ occupy the favored areas of the Ramachandran plot. The conformations of the majority of the residues are well defined, except for a few terminal residues and the loop regions between α-helices.

The monomers associate into a dimer through the formation of an antiparallel coiled coil between the α2 helices, and domain swapping (an exchange of secondary structural elements between constituent monomers (M. J. Bennett, M. P. Schlunegger, D. Eisenberg, Protein Sci 4, 2455-68 (1995)) of two α1 helices, where one α1 helix swings back and packs against the α2 helix from the second monomer (FIG. 2A, FIG. 2B). The four helices in the dimer lie approximately in one plane. The intertwined packing arrangement gives rise to an extensive dimer interface with a total of 217 van der Waals contacts (radius cutoff=4 Å) 7 salt bridges, and 3 hydrogen bonds.

More than half of the dimer interference lies within the hydrophobic core in the antiparallel coiled coil. Most of the a and d residues (see FIG. 1 description)—including Ile31, Leu35, Ile42, Leu45, Val49, Met56, Leu59, and Leu63—are hydrophobic and pack in a typical “knobs-in-holes” mode (F. H. C. Crick, Acta Crystallogr 6, 689-97 (1953); E. K. O'Shea, J. D. Klemm, P. S. Kim, T. Alber, Science 254:539-44 (1991)). The exception is Glu52 (d); this charged residue is stabilized by forming an intra-chain salt bridge with Arg55 (g). Apart from the hydrophobic core in the coiled-coil interface, there is a preliminary aromatic core between the α1 helix of one chain (including Phe7, Trp11, and Phe15) and the α2-helix of the other chain (including Phe54 and Tur58) (FIG. 2A inset). This exchange of α1-helices buries a hydrophobic patch on the outer surface of the coiled coil and provides additional structural stability. Coiled-coil oligomerization domains typically assemble in a parallel fashion with only a few known exceptions (for example, see S. M. Soisson, B. MacDougall-Shackleton, R. Schleif, C. Wolberger, Science 276: 421-5 (1997); H. J. Zuccola, J. E. Rozzelle, S. M. Lemon, B. W. Erickson, J. M Hogle, Structure 6:821-30 (1998)). It is possible that the swapping of α1-helices plays a role in stabilizing the antiparallel orientation. The structure described herein reveals an efficient way of assembling dimers and providing improved stability by using a hybrid of two distinct oligomerization modes: antiparallel coiled-coil packing and domain swapping, both recognized as important mechanisms for forming oligomeric proteins (M. J. Bennett, M. P. Schlunegger, D. Eisenberg, Protein Sci 4, 2455-68 (1995); C. Cohen, D. A. Parry, Science 263, 488-9 (1994)).

Two dimers associate into a tetramer with an approximately 130° crossing angle between the dimer coiled-coil axes (FIG. 3A; FIG. 3B), in which the dimer-dimer interface is significantly less polar than the solvent-exposed surface (FIG. 4A; FIG. 4B). The physiological significance of tetramer formation, however, remains to be determined. Previous studies have shown that substitution of the Bcr oligomerization domain with the GCN4 dimeric parallel coiled coil also activated kinase activity (J. R. McWhirter, J. Y. Wang, Oncogene 15, 1625-34 (1997)). In addition, different dimerization motifs, such as the helix-loop-helix domain in Tel, can activate Abl and lead to a different type of leukemia (P. Padaopoulos, S. A. Ridge, C. A. Boucher, C. Stocking, L. M. Wiedemann, Cancer Res 55, 34-8 (1995). These results support the notion that the mere clustering of kinase domains, mediated by dimerization, is sufficient to induce activation in a way similar to the activation of receptor tyrosine kinases by ligands (J. Schlessinger, Cell 103, 211-25 (2000)). However, the crystal structure indicates how a tetramer is likely to be more efficient than a dimer in bringing downstream domains together. The distance between the two C-termini in the dimer is approximately 60 Å, whereas tetramer formation brings opposite C-termini across the dimer-dimer interface into a closer proximity of approximately 24 Å.

The coordinates for Bcr₁₇₂ have been deposited in the Protein Data Bank (Accession Code 1K1F).

TABLE
Data collection, phasing, and refinement statistics
Data collection (20.0-2.2 A)

| Data set | λ (Å) | % complete | Rsym¹ (%) |
|---|---|---|---|
| SeMet λ1 | 0.9793 | 99.2 | 5.4 |
| SeMet λ2 | 0.9789 | 99.5 | 6.0 |
| SeMet λ3 | 0.9684 | 99.3 | 6.2 |

Phasing statistics(2.0-2.6 A)

| | Rcullis² | Rcullis² | Rcullis² | Phasing power³ | Phasing power³ |
|---|---|---|---|---|---|
| Derivative | Acentric | Centric | Anomalous | Acentric | Centric |
| SeMet λ2 vs. λ1 | 0.85 | 0.90 | 0.62 | 0.91 | 0.58 |
| SeMet λ3 vs. λ1 | 0.67 | 0.67 | 0.67 | 1.59 | 1.12 |

Overall figure of merit (before solvent flattening): 0.62.

Refinement statistics (20.0-2.2 A)

| Nonhydrogen | | Number of reflections | | | | r.m.s. deviations | |
|---|---|---|---|---|---|---|---|
| protein atoms | Waters | working | free | Rcryst⁴ | Rfree⁴ | bonds (Å) | angles (°) |
| 4366 | 422 | 48,746 | 2505 | 26.2% | 29.5% | 0.008 | 1.42 |

¹Rsym = ΣΣj11j − <|> / Σ Σ |<|>|, where 1j is the recorded intensity of the reflection j and <|> is the mean recorded intensity over multiple recordings.
²Rculli = Σ||F(λ1) ± F(λ1)1 − 1F_{h(λ1),c}||/Σ|F(λi) ± F(λ1)1, where F_{h(λi),c} is the calculated heavy atom structure factor.
³Phasing power = <F_{h(λ1)}>/E, where <F_{h(λi)}> is the root-mean-square (r.m.s.) heavy atom structure factor and E is the residual lack-of-closure error.
⁴Rcryst, Rfree = Σ||F_{obs}| − |F_{calc}||/|F_{obs}|, where the crystallographic and free R factors are calculated using the working and free reflection sets, respectively.

### EXAMPLE 3

### Determining Inhibition of Bcr-Abl Oligomerization

Whether the Bcr-Abl oligomerization domain inhibits full-length Bcr-Abl oligomerization in a dominant-negative manner was assessed. The N-terminal 160 amino acids of Bcr, which contain the oligomerization domain, as well as the full-length Bcr, inhibit Bcr-Abl transformation (X. Y. Guo, et al., Oncogene 17, 825-33 (1998); Y. Wu, et al., Oncogene 18, 4416-24 (1999). As shown herein, the Bcr oligomerization domain alone, for example, as contained in Bcr₁₋₇₄ was sufficient to inhibit Bcr-Abl function. In a focus-formation assay with Rat-1 fibroblasts, coexpression of the oligomerization domain inhibited the transforming potential of Bcr-Abl (FIG. 5). Rat-1 fibroblasts were contransfected with MIG-P210 (a retroviral vector expressing full-length Bcr-Abl) or MIG (vector control) and increasing doses of plasmid B74 (a retroviral vector expressing Bcr₁₋₇₄). The total number of transformed foci was quantified after 3 weeks. Transected cultures were stained with 0.4% crystal violet to better visualize transformed foci. This demonstrates the therapeutic potential of inhibiting Bcr-Abl oligomerization in reducing proliferation and oncogenesis of Bcr-Abl-expressing cells.

For expression in mammalian cells, Bcr₁₋₇₄ fragments were PCR amplified from pMBP/Bcr₁₋₇₄ (with the optimal E. coli codons) as mentioned above, and inserted into the retroviral expression vector pMX-IRES-GFP (X. Liu et al., Anal Biochem 280, 20-28 (2000)) using BamHI and NotI sites. The resulting plasmid was denoted p74-IES-GFP (B74). Although it has been reported that the Bcr oligomerization domain as contained in residues 1-71 (SEQ ID NO: 19; SEQ ID NO: 20) of the Bcr protein cannot be stably expressed in mammalian cells (C. L. Sawyers, N Engl J Med 340, 1330-40 (1999)), a fair amount of Bcr₁₋₇₄ (SEQ ID NO: 7) protein was detected by Western blotting when B74 was expressed in vivo. It is possible that use of E. coli-optimized codons improved expression in this domain. In a focus-formation assay, Rat-1 cells (5×10⁵) were cotransfected with mPSCV-P210-IRE S-GFP (MIG-P210) or pMSCV-IRES-GFP (MIG) alone and in different amounts of p74-IRES-GPF (B74) by calcium phosphate transfection and glycerol shock. The total number of transformed foci was counted after 3 weeks.

Both the kinase and oligomerization domains are essential for Bcr-Abl oncogenicity and they likely independently serve as drug targets. A specific kinase inhibitor has been shown to target the Abl kinase domain (T. Schindler, et al., Science 289, 1938-42 (2000)) and has been clinically effective in the treatment of CML (M. J. Mauro, B. J. Druker, Curr Oncol Rep 3, 223-7 (2001). However, drug-resistant leukemic cells have been observed in CML patients following drug treatment (M. E. Gorre, et al., Science 21, 21 (2001). Targeting the Bcr oligomerization domain may provide an alternative or complementary therapeutic approach to specifically disrupt Bcr-Abl transformation potential.

The crystal structure provides a template for the rational design of inhibitors disrupting Bcr-Abl oligomerization.

While this invention has been particularly shown and described with references to preferred embodiments thereof, it will be understood by those skilled in the art that various changes in form and details may be made therein without departing from the scope of the invention encompassed by the appended claims.

The teachings of all the references, patents and patent applications cited throughout this application are hereby incorporated by reference in their entirety.

What is claimed is:

1. An isolated Bcr-Abl oligomerization domain polypeptide that forms a stable α-helical structure.

2. The isolated Bcr-Abl oligomerization domain polypeptide of claim 1 that has the amino acid sequence of SEQ ID NO: 1.

3. The isolated Bcr-Abl oligomerization domain polypeptide of claim 1 that is a Bcr₁₋₇₂ sequence.

4. The Bcr₁₋₇₂ of claim 3 that has the amino acid sequence of SEQ ID NO.: 3.

5. The Bcr₁₋₇₂ of claim 3 that has the amino acid sequence of SEQ ID NO.: 5.

6. The isolated Bcr-Abl oligomerization domain polypeptide of claim 1 that has the amino acid sequence of SEQ ID NO: 7.

7. The isolated Bcr-Abl oligomerization domain polypeptide of claim 1 that has the amino acid sequence of SEQ ID NO: 9.

8. An isolated nucleic acid encoding a Bcr-Abl oligomerization domain that forms a stable α-helical structure.

9. The nucleic acid of claim 8 that encodes an amino acid sequence of SEQ ID NO: 1.

10. The nucleic acid of claim 8 that encodes a Bcr₁₋₇₂ sequence.

11. The nucleic acid of claim 10 that encodes the amino acid sequence of SEQ ID NO.: 3.

12. The nucleic acid of claim 10 that encodes the amino acid sequence of SEQ ID NO.: 5.

13. The nucleic acid of claim 8 that encodes the amino acid sequence of SEQ ID NO: 7.

14. The nucleic acid of claim 8 that encodes the amino acid sequence of SEQ ID NO: 9.

15. The isolated nucleic acid of claim 8 that has the nucleotide sequence of SEQ ID NO: 2.

16. The isolated nucleic acid of claim 10 that has the nucleotide sequence of SEQ ID NO: 4.

17. The isolated nucleic acid of claim 10 that has the nucleotide sequence of SEQ ID NO: 6.

18. The isolated nucleic acid of claim 8 that has the nucleotide sequence of SEQ ID NO: 8

19. The isolated nucleic acid of claim 8 that has the nucleotide sequence of SEQ ID NO: 10.

20. An expression vector comprising the nucleic acid of claim 8.

21. An expression vector comprising a nucleic acid selected from the group consisting of SEQ ID NO: 2, SEQ ID NO: 4, SEQ ID NO: 6, SEQ ID NO: 8 and SEQ ID NO: 10.

22. A host cell comprising the expression vector of claim 20.

23. A method of producing a Bcr-Abl oligomerization domain polypeptide comprising culturing the host cell of claim 22 under conditions suitable for producing a polypeptide.

24. The method of claim 23, further comprising isolating the Bcr-Abl oligomerization domain polypeptide.

25. A method of identifying an agent which binds to a Bcr-Abl oligomerization domain that forms a stable α-helical structure comprising contacting an isolated Bcr-Abl oligomerization domain that forms a stable α-helical structure with a candidate agent and detecting the resulting domain-agent complex, wherein formation of a domain-agent complex is indicative of binding to a Bcr oligomerization domain that forms a stable α-helical structure.

26. The method of claim 25, wherein the agent is selected from the group consisting of a protein, polypeptide, peptidomimetic, prodrug, binding agent, antibody, small molecule or other drug, or ribozyme.

27. The method of claim 25, wherein the isolated Bcr-Abl oligomerization domain has the amino acid sequence of SEQ ID NO: 1.

28. The method of claim 25, wherein the isolated Bcr-Abl oligomerization domain is of a Bcr₁₋₇₂ sequence.

29. The method of claim 28, wherein the Bcr₁₋₇₂ has the amino acid sequence of SEQ ID NO.: 3.

30. The method of claim 28, wherein the Bcr₁₋₇₂ sequence has the amino acid sequence of SEQ ID NO.: 5.

31. The method of claim 25, wherein the isolated Bcr-Abl sequence oligomerization domain has the amino acid sequence of SEQ ID NO: 7.

32. The method of claim 25, wherein the isolated Bcr-Abl oligomerization domain has the amino acid sequence of SEQ ID NO: 9.

33. An agent identified by the method of claim 25.

34. A method of identifying an inhibitor of Bcr-Abl oligomerization, comprising:
a) introducing Bcr-Abl and a candidate inhibitor into cells;
b) maintaining the cells under conditions appropriate for Bcr-Abl transformation of the cells to occur; and
c) comparing the extent to which transformation of the cells occurs in the presence of the candidate inhibitor to the extent to which transformation occurs in the absence of the candidate inhibitor,
wherein if the cells are transformed to a lesser extent in the presence of the inhibitor, the candidate inhibitor is an inhibitor of Bcr-Abl oligomerization.

35. The method of claim 34, wherein the inhibitor is selected from the group consisting of a protein, polypeptide, peptidomimetic, prodrug, binding agent, antibody, small molecule or other drug, or ribozyme.

36. The method of claim 34, wherein the Bcr-Abl oligomerization domain forms a stable α-helical structure.

37. The method of claim 34, wherein the isolated Bcr-Abl oligomerization domain has the amino acid sequence of SEQ ID NO: 1.

38. The method of claim 34, wherein the isolated Bcr-Abl oligomerization domain is a Bcr₁₋₇₂ sequence.

39. The method of claim 38, wherein the Bcr₁₋₇₂ has the amino acid sequence of SEQ ID NO.: 3.

40. The method of claim 38, wherein the Bcr₁₋₇₂ has the amino acid sequence of SEQ ID NO.: 5.

41. The method of claim 34, wherein the isolated Bcr-Abl oligomerization domain has the amino acid sequence of SEQ ID NO: 7.

42. The method of claim 34, wherein the isolated Bcr-Abl oligomerization domain has the amino acid sequence of SEQ ID NO: 9.

43. The method of claim 34, wherein the cells are mammalian cells.

44. An agent identified by the method of claim 34.

45. A method of treating or preventing a disease associated with Bcr-Abl oligomerization in a subject comprising administering to a subject in need thereof an effective amount of an inhibitor of a Bcr-Abl oligomerization domain.

46. The method of claim 45, further comprising administering a tyrosine kinase inhibitor.

47. A method of treating or preventing chronic myelongenous leukemia, CML, in a subject comprising administering to a subject in need thereof an effective amount of an inhibitor of a Bcr-Abl oligomerization domain.

48. A method of treating or preventing acute lymphoblastic anemia, ALL, in a subject comprising administering to a subject in need thereof an effective amount of an inhibitor of a Bcr-Abl oligomerization domain.

49. A method of preparing a medicament for use in treating or preventing a disease associated with Bcr-Abl oligomerization, the medicament comprising an inhibitor of a Bcr-Abl oligomerization domain.

50. The method of claim 49, wherein the disease is CML.

51. The method of claim 49, wherein the disease is ALL.

52. The method of claim 49, wherein the medicament further comprises a tyrosine kinase inhibitor.
